(19) United States
(12) Patent Application Publication
(10) Pub. No.: US 2007/0049966 A1
(43) Pub. Date: Mar. 1, 2007

(54) Surgical instrument

(76) Inventors: Frank Bonadio, Bray (IE); John Butler, Blackrock (IE); Trevor Vaugh, Birr (IE); Catherine Deegan, Clontarf (IE); Shane Joseph MacNally, Bray (IE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/386,103

(22) Filed: Mar. 22, 2006

Related U.S. Application Data
(60) Provisional application No. 60/663,732, filed on Mar. 22, 2005.
(60) Provisional application No. 60/699,365, filed on Jul. 15, 2005.

(51) Int. Cl. A61B 17/00

(52) U.S. Cl. 606/206

(57) ABSTRACT

A surgical access system (100) comprises an access port (5), a rigid cannula having a shaft (11) and a laparoscopic surgical instrument (101). The access port (5) comprises a seal (6) and a retractor. The retractor comprises a distal O-ring (71), an outer proximal ring member (77), an inner proximal ring member (78) and a sleeve (72). The sleeve (72) extends distally from the inner proximal ring member (78) to the distal O-ring (71) in a first layer, is looped around the distal O-ring (71), and extends proximally in a second layer between the inner proximal ring member (78) and the outer proximal ring member (77). The instrument (101) comprises a shaft (103) with a rigid proximal region (104), a flexible intermediate region (105), and a rigid distal region (106). The instrument shaft (103) may be inserted through the cannula shaft (11). The instrument (101) has a rigid end effector (107) releasably coupled to the distal end (108) of the instrument shaft (103). An actuator (109) for actuating the end effector (107) is provided at the proximal end (110) of the instrument shaft (103). The actuator (109) is movable along the instrument shaft (103) parallel to the longitudinal axis of the instrument shaft (103).

Accessing the abdominal cavity while preserving the abdominal wall as much as possible is the aim of any surgical or exploratory procedure. Retraction devices have been used to this end. A retractor can help to expose an operative site and minimise the incision required to carry out the operation.

Minimally invasive surgery is an evolving surgical method that attempts to reduce the size of incisions required, in many cases dramatically. By using a so-called “keyhole” or cannula, the surgeon can gain access with instruments into the abdominal cavity to carry out an operation through a very small series of holes in the abdominal wall. Unlike in the case of “open surgery”, primary retraction then must be accomplished by lifting the abdominal wall away from the abdominal viscera. This is most often accomplished with the use of gas in a technique known as insufflation.

The use of a cannula to gain access as a means to see inside the abdomen or introduce surgical instruments has existed since the late 19^{th} century. A cannula comprises a rigid tube, which is inserted through the abdominal wall and is held in place by the tension of the abdominal wall itself around the inserted cannula. The tube must accommodate various thicknesses of abdominal wall and extend significantly both inside and outside the abdomen to avoid slipping out of the incision, and thereby causing gas pressure to escape.

The basic construction of a cannula, however, presents significant limitations in carrying out a surgical procedure. Some of these limitations are as follows.
1. A cannula is held in place, and thus prevents the escape of gas, by tissue tension. This tension can vary depending on the way the cannula is introduced or weaken during the operation under normal surgical manipulation.
2. A cannula extends significantly into the abdominal cavity taking up precious space and interfering with other instruments.
3. A cannula restricts the movement of instruments as they are rigid structures.
4. A rigid cannula presents significant limitations on the design of the instrument which must be passed through the cannula.
5. A cannula takes up a significant space outside of the abdomen, shortening the effective length, and therefore reach, of the surgical instrument.

This invention is aimed at addressing at least some of these problems.

### Statements of Invention

According to the invention there is provided a surgical device comprising a shaft, the shaft comprising a distal section, a proximal section, and a bend section located between the distal section and the proximal section.

In one embodiment of the invention the distal section is substantially parallel to the proximal section. The distal section and proximal section may be substantially co-axial. The distal section and the proximal section may be substantially offset.

In one case the distal section is substantially straight. The proximal section may be substantially straight. The bend section may be at least partially of curved shape. The bend section may be at least partially of arcuate shape.

In one embodiment the bend section is pre-set. The shaft may be at least partially rigid. A distal region of the shaft adjacent a distal end of the shaft may be rigid. A proximal region of the shaft adjacent a proximal end of the shaft may be rigid. The shaft may be at least partially malleable. The shaft may be at least partially flexible. An intermediate region of the shaft intermediate the proximal region and the distal region may be flexible.

In another embodiment the shaft defines a lumen extending therethrough. The surgical device may comprise a cannula.

In one case the surgical device comprises an instrument. The surgical device may comprise an end effector at a distal end of the shaft. The end effector may be releasably coupled to the distal end of the shaft. The radial dimension of the end effector may be substantially greater than the radial dimension of the shaft. The end effector may be movable between an open configuration and a closed configuration. The end effector may be movable relative to the shaft. The end effector may be translatable relative to the shaft. The end effector may be rotatable relative to the shaft. The end effector may be translatable and rotatable relative to the shaft.

In one case the surgical device comprises an actuator for actuating the end effector. The actuator may be movable between an end effector open configuration and an end effector closed configuration. The actuator may be biased towards an end effector open configuration. The actuator may be movable along the shaft. The actuator may be movable parallel to the longitudinal axis of the shaft. The actuator may comprise a plunger.

In another case the surgical device comprises a coupling member to couple the actuator to the end effector. The coupling member may comprise at least one tubular element. The tubular element may extend between the actuator and the end effector. The coupling member may comprise a first tubular element extending from the actuator and a second tubular element extending from the end effector. The coupling member may comprise a universal joint to couple the first tubular element to the second tubular element. The shaft may define a lumen extending therethrough. The coupling member may be at least partially located within the lumen.

The invention provides in one case a laparoscopic surgical device.

In another aspect of the invention there is provided a surgical instrument comprising a shaft, an end effector at a distal end of the shaft, and an actuator for actuating the end effector, the actuator being movable along the shaft between an end effector open configuration and an end effector closed configuration.

In one embodiment of the invention the actuator is movable parallel to the longitudinal axis of the shaft. The actuator may comprise a plunger. The actuator may be biased towards an end effector open configuration. The end effector may be releasably coupled to the distal end of the shaft. The end effector may be movable between an open configuration and a closed configuration. The end effector may be movable relative to the shaft. The end effector may be translatable relative to the shaft. The end effector may be rotatable relative to the shaft. The end effector may be translatable and rotatable relative to the shaft.

In one case the shaft is at least partially flexible. The shaft may be at least partially malleable. The shaft may be at least partially rigid. A distal region of the shaft adjacent a distal end of the shaft may be rigid. A proximal region of the shaft adjacent a proximal end of the shaft may be rigid. An intermediate region of the shaft intermediate the proximal region and the distal region may be flexible.

In one embodiment the shaft of the instrument is configured for insertion through a surgical device shaft.

In a further aspect the invention provides a surgical instrument comprising a shaft and an end effector at a distal end of the shaft, the radial dimension of the end effector being substantially greater than the radial dimension of the shaft.

In one case the invention provides a laparoscopic surgical instrument.

The invention also provides in another aspect a surgical access system comprising:
a surgical access port configured for location adjacent to an incision; and
a surgical device of the invention for insertion through the access port.

In one aspect of the invention the access port comprises an access valve or seal, through which the surgical device is insertable. The access valve or seal may comprise a gelatinous elastomeric material for receiving the surgical device. The access valve or seal may have a pin hole therein to receive the surgical device.

In one case the access port comprises a retractor. The access valve or seal may be mounted or mountable to the retractor. The retractor may comprise:
a distal anchoring member for insertion through an incision; and
an elongate member extending proximally from the distal anchoring member.

The retractor may comprise a proximal ring for location externally of an incision, with the elongate member extending between the distal anchoring member and the proximal ring. The distal anchoring member may comprise a distal ring. The distal ring may be formed from an elastomeric material. The elongate member may comprise a sleeve. The elongate member may comprise a single material layer. At least a portion of the elongate member may comprise two material layers. The elongate member may be wrapped around the distal anchoring member. The elongate member may be fixed to the proximal ring at one end, and the elongate member may extend from the proximal ring to the distal anchoring member to define an inner material layer, and the elongate member may extend from the distal anchoring member to the proximal ring to define an outer material layer. The elongate member may be slidingly received over a portion of the proximal ring. The proximal ring may comprise an inner proximal ring member and an outer proximal ring member between which the elongate member is led.

In another embodiment the surgical device comprises a first surgical instrument. The surgical device may comprise a first end effector at a distal end of the shaft.

In one case the system comprises a second surgical device for insertion through the access port. The second surgical device may comprise a shaft comprising a bend section. The second surgical device may comprise a second surgical instrument. The second surgical device may comprise a second shaft, and a second end effector at a distal end of the second shaft. The first end effector and the second end effector may be of the same type of end effector. The first end effector and the second end effector may be different types of end effectors.

In another embodiment the system comprises a third surgical device for insertion through the access port. At least one of the surgical devices may comprise a laparoscope.

In one embodiment the system comprises a surgical instrument comprising a shaft, the instrument shaft being insertable through the surgical device shaft. The instrument may comprise an instrument of the invention.

In one case the invention provides a laparoscopic surgical access system.

In a further aspect of the invention there is provided a method of performing a surgical procedure, the method comprising the steps of:
creating a wound opening;
inserting a surgical device at least partially through the wound opening to access the wound interior;
applying a manipulating action to the surgical device to manipulate the surgical device into a desired position and/or orientation within the wound interior; and
releasing the manipulating action, the surgical device substantially maintaining the desired position and/or orientation within the wound interior after release of the manipulating action.

In one embodiment of the invention the manipulating action comprises a manipulating force to manipulate the surgical device into a desired position. The manipulating action may comprise a manipulating torque to manipulate the surgical device into a desired orientation. The method may comprise the step of sealing the wound opening. The method may comprise the step of retracting the wound opening.

In one case the surgical device comprises a surgical instrument. The surgical device may comprise a shaft, the shaft comprising a bend section.

According to the invention there is provided a system comprising:
an access port comprising an access valve or seal for location adjacent to an incision; and
a surgical device having a shaft for location in the valve adjacent to the incision, the shaft having a bend therein.

In one embodiment the shaft comprises a distal section and a proximal section and the bend is located between the distal and proximal sections. The distal section may be substantially parallel to the proximal section. The distal and proximal sections may be substantially co-axial. Alternatively the distal and proximal sections are substantially offset.

In one embodiment the bend is at least partially of curved shape. The bend may be at least partially of arcuate shape.

In one embodiment the bend in the shaft is pre-set.

The shaft may be at least partially malleable or at least partially flexible.

In one embodiment the shaft defines a lumen extending therethrough. The surgical device may comprise a cannula.

In one case the system comprises an instrument having a shaft, the instrument shaft being insertable through the surgical device shaft. The instrument shaft may be at least partially flexible. The instrument shaft may be at least partially malleable. The instrument shaft may be at least partially rigid.

In one embodiment a distal region of the instrument shaft adjacent a distal end of the instrument shaft is rigid. A proximal region of the instrument shaft adjacent a proximal end of the instrument shaft may be rigid. An intermediate region of the instrument shaft intermediate the proximal and distal regions may be flexible.

In another case the instrument comprises an end effector at a distal end of the instrument. The end effector may be releasably coupled to the distal end of the instrument shaft. The instrument may comprise an actuator for actuating the end effector. The actuator may be movable between an end effector open configuration and an end effector closed configuration. The actuator may be biased towards the end effector open configuration. The actuator may be movable along the instrument shaft. The actuator may be movable parallel to the longitudinal axis of the instrument shaft. The actuator may comprise a plunger.

The system may comprise a second surgical device for insertion through the access port. The second device comprise a shaft having a bend therein.

The system may comprise a third surgical device for insertion through the access port.

In one embodiment at least one of the surgical devices comprises a laparoscope.

The access valve or seal may comprise a gelatinous elastomeric material for receiving the surgical device. The access valve or seal may have a pin hole therein to receive a surgical device.

In one embodiment the access port comprises a retractor to which the access valve or seal is mounted or mountable.

In one arrangement the retractor comprises:
a distal anchoring member; and
an elongate member extending proximally from the distal anchoring member;

In one case the elongate member comprises a sleeve.

The sleeve may comprise a single material layer or at least a portion of the sleeve may comprise two material layers.

In one embodiment the sleeve is wrapped around the distal anchoring member.

The distal anchoring member may comprise a distal ring which may be formed from an elastomeric material.

In one embodiment the retractor comprises:
a distal ring;
a proximal ring; and
a sleeve having a portion between the distal ring and the proximal ring that includes two material layers.

The sleeve may be fixed to the proximal ring at one end, and the sleeve may extend from the proximal ring to the distal ring to define the inner material layer, and the sleeve may extend from the distal ring to the proximal ring to define an outer material layer. The sleeve may be slidingly received over a portion of the proximal ring.

In one embodiment the proximal ring comprises an inner proximal ring member and an outer proximal ring member between which the sleeve is led.

The invention also provides surgical device having a shaft, the shaft having a bend therein located between a distal section and a proximal section of the shaft.

The distal section may be substantially parallel to the proximal section. In one case the distal and proximal sections are substantially co-axial. Alternatively the distal and proximal sections are substantially offset.

The bend may be at least partially of curved shape. The bend may be at least partially of arcuate shape.

In one embodiment the bend in the shaft is pre-set. The shaft may be at least partially malleable or at least partially flexible.

In one embodiment the shaft defines a lumen extending therethrough. The surgical device may comprise a cannula.

In a further aspect, the invention provides an instrument having a shaft, the instrument shaft being insertable through a surgical device shaft.

In one embodiment the instrument shaft is at least partially flexible. The instrument shaft may be at least partially malleable. The instrument shaft may be at least partially rigid.

In one case a distal region of the instrument shaft adjacent a distal end of the instrument shaft is rigid. A proximal region of the instrument shaft adjacent a proximal end of the instrument shaft may be rigid. An intermediate region of the instrument shaft intermediate the proximal and distal regions may be flexible.

In another embodiment the instrument comprises an end effector at a distal end of the instrument. The end effector may be releasably coupled to the distal end of the instrument shaft. The instrument may comprise an actuator for actuating the end effector. The actuator may be movable between an end effector open configuration and an end effector closed configuration. The actuator may be biased towards an end effector open configuration. The actuator may be movable along the instrument shaft. The actuator may be movable parallel to the longitudinal axis of the instrument shaft. The actuator may comprise a plunger.

The incision may be a laparoscopic incision. The sides of the incision may be retracted to a diameter of less than 40 mm, preferably to a diameter of between 3 mm and 35 mm, typically to a diameter of about 15 mm to 20 mm.

The instrument may be a laparoscopic instrument which may have a diameter of less than 40 mm, typically the instrument has a diameter of between 3 mm and 35 mm, in one case the instrument has a diameter of less than 10 mm.

### BRIEF DESCRIPTION OF THE DRAWINGS

The invention will be more clearly understood from the following description of some embodiments thereof, given by way of example only, with reference to the accompanying drawings, in which:

FIG. 1 is a cross sectional, side view of a surgical instrument access system according to the invention, in use;

FIG. 2 is a view similar to FIG. 1 of the surgical instrument access system showing different positions for a surgical instrument;

FIGS. 3(a) to 3(c) are elevational views of the shafts of various surgical instruments according to the invention;

FIG. 4 is a cross sectional, side view of one instrument in use with the surgical instrument access system of FIG. 1;

FIGS. 5(a) and 5(b) are partially cross-sectional, side views of another instrument according to the invention in different positions of use;

FIG. 6 is a partially cross-sectional, side view of another surgical instrument access system according to the invention with two instruments;

FIGS. 7(a) and 7(b) are cross-sectional, side views of a two surgical instrument access system according to the invention with the instruments in different configurations;

FIG. 8 is a partially cross-sectional, side view of another two surgical instrument access system according to the invention;

FIG. 9 is a partially cross-sectional, side view of a further two surgical instrument access system according to the invention;

FIG. 10 is a partially cross-sectional, side view of a still further two surgical instrument access system according to the invention;

FIG. 11 is a partially cross-sectional, side view of another surgical instrument access system according to the invention having three instruments;

FIGS. 12(a) to 12(c) are partially cross-sectional, side views of another surgical instrument access port system according to the invention;

FIG. 13 is a partially cross-sectional, side view of a further surgical instrument access port system according to the invention;

FIG. 14(a) is a partially cross-sectional, side view of an access port system of the prior art for comparative purposes;

FIG. 14(b) is a partially cross-sectional, side view of another surgical instrument access port system according to the invention;

FIGS. 15(a) and 15(b) are cross-sectional, side views of a conventional system;

FIGS. 16(a) and 16(b) are elevational views of a surgical instrument according to the invention in different configurations of use;

FIG. 17 is a perspective view of a surgical device and a surgical instrument according to the invention;

FIGS. 18 and 19 are partially cross-sectional, side views of the surgical instrument of FIG. 17 inserted through the surgical device of FIG. 17, with the surgical device located in a surgical access port according to the invention;

FIGS. 20 and 21 are perspective views of the surgical instrument of FIG. 17 inserted through the surgical device of FIG. 17;

FIG. 22 is a side view of another surgical device according to the invention;

FIG. 23 is an enlarged, partially cross-sectional, side view of a port of the surgical device of FIG. 22;

FIG. 24 is a view similar to FIG. 23 of another surgical device according to the invention;

FIGS. 25 and 26 are partially cross-sectional, side views of the surgical device of FIG. 22, in use;

FIGS. 27 and 28 are views similar to FIGS. 25 and 26 of a conventional instrument, in use;

FIG. 29 is another view similar to FIGS. 25 and 26 of the surgical device of FIG. 22, in use;

FIG. 30 is a side view of another surgical device according to the invention;

FIG. 31 is a partially cross-sectional, side view of the surgical device of FIG. 30, in use;

FIG. 32 is a view similar to FIG. 31 of the surgical device of FIG. 30 and another surgical device according to the invention, in use; and

FIG. 33 is a view similar to FIG. 31 of another surgical device according to the invention, in use.

### DETAILED DESCRIPTION

Referring to the drawings there are illustrated various laparoscopic surgical instrument access systems of the invention for an incision 1, for example in an abdominal wall 2. The construction of the various components and their attributes will be explained in detail below.

The instrument access systems of the invention generally comprise an access port 5 having an access valve or seal 6 for location adjacent to the incision 1. The system also comprises a laparoscopic surgical device comprising a cannula or an instrument 10 having a shaft 11 for location in the valve or seal 6 adjacent to the incision 1. The device of the invention has a bend section 15 therein. The device may be a visualisation tool such as a camera, light, or a laparoscope and/or may have any suitable end effector such as a grasper, scissors, stapler or the like.

It will be noted that the valve/seal 6 has a very low profile, especially with respect to the inside of the incision 1. The devices are positively retained in the incision 1 against pull-out forces. This is in contrast to a conventional cannula, in which the rigid tube of the cannula must be extended significantly into the abdomen to ensure that it remains anchored in the abdomen, otherwise gas pressure may cause it to become dislodged. In conventional systems, because of the cannula length extending into the abdomen, the shaft of the instrument cannot be steered until the steerable section has exited the cannula. Thus, there are severe limitations on the use of such instruments using a conventional cannula. These problems are overcome at least in part using the systems of the invention.

The access port 5 comprises a liner and/or retractor. The liner/retractor part comprises a distal anchoring member 71 and an elongate member 72 extending proximally of the distal anchoring member 71. In this case, the elongate member is provided in the form of a sleeve 72 of flexible, polymeric film material which lines the sides of the wound opening 1, in use. The distal anchoring member 71 in this case comprises a resilient O-ring. In use, a relatively small incision 1 is made in the abdominal wall 2 to form the wound opening. A typical length for the incision 1 is in the range of from 12 mm to 30 mm. The resilient distal O-ring 71 is then manipulated into an elongate, oblong shape by squeezing the distal O-ring 71 to facilitate insertion of the distal O-ring 71 through the wound opening 1, until the distal O-ring 71 is fully located within the abdominal cavity and the sleeve 72 lines the wound opening 1. The sleeve 72 is then pulled upwardly to cause the distal O-ring 71 to engage with the internal surface of the abdominal wall.

Any suitable valve or seal or combinations of valves and/or seals may be provided for the instrument 11. Such valve or valves are generically indicated by the reference numeral 6 in the drawings.

The sleeve 72 may be a single layer sleeve or may have two layers at least in the section which lines the wound opening 1. In one such arrangement the sleeve 72 is wrapped around the distal ring 71 and has an outer layer 74 which lines the wound opening 1 and an inner layer 75. A clamp is provided, in this case a proximal clamp, comprising an outer proximal ring member 77 and an inner proximal ring member 78 between which the sleeve 72 extends. In this case the inner proximal clamp 78 is mounted to or provided by part of a housing for the valve 6. The sleeve 72 is mounted at one end to the ring member 78 or housing and extends to form the inner layer 75, is wrapped around the distal ring 71 and extends to form the outer layer 74. The sleeve 72 is in this case slidable on at least portion of the inner proximal clamp ring 78. On pulling of the sleeve 72 upwardly the wound opening 1 is retracted. Because of the sleeve pathway a free end of the sleeve 72 is external of the valve 6 and can be readily removed, if desired.

It will be appreciated that the access port may be provided in any suitable form, for example the access port may be provided in the form of one or more of the devices described in International patent application No. PCT/IE2003/000141, the relevant contents of which are incorporated herein by reference, and/or in the form of one or more of the devices described in International patent application No. PCT/IE2005/000113, the relevant contents of which are incorporated herein by reference.

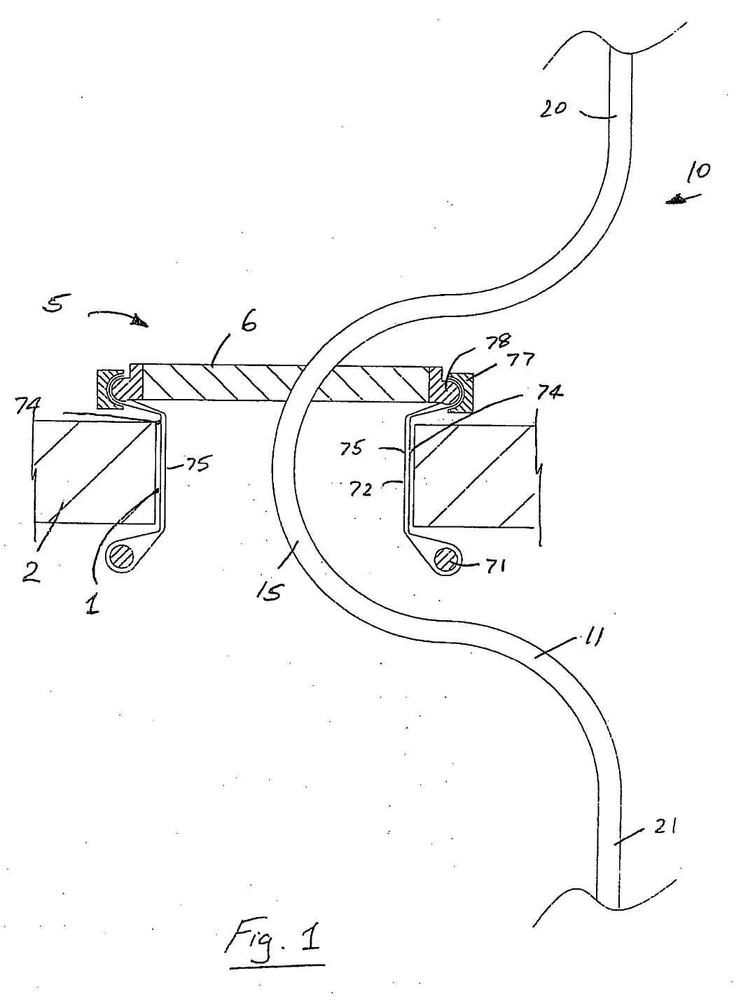

Referring to FIG. 1, in one embodiment of the invention the surgical device shaft 11 comprises a straight proximal section 20 and a straight distal section 21, and the bend section 15 is intermediate the distal and proximal sections 20, 21. In this case the distal section 21 is substantially parallel to the proximal section 20 and may be co-axial therewith. This arrangement ensures that the distal end of the instrument 11 travels in the same direction that the surgeon moves the proximal end.

The access port 5 has a very low profile. In particular, when deployed the distal ring 71 is located close to the interior surface of the abdominal wall 2, and thus the access port 5 only extends distally into the abdominal cavity by a very small distance. It is therefore possible to access a very large space within the abdominal cavity using the bent shaft 11. In particular the bend 15 may be located close to the distal ring 71 to access part of the abdominal cavity laterally of the incision 1, as illustrated in FIG. 1.

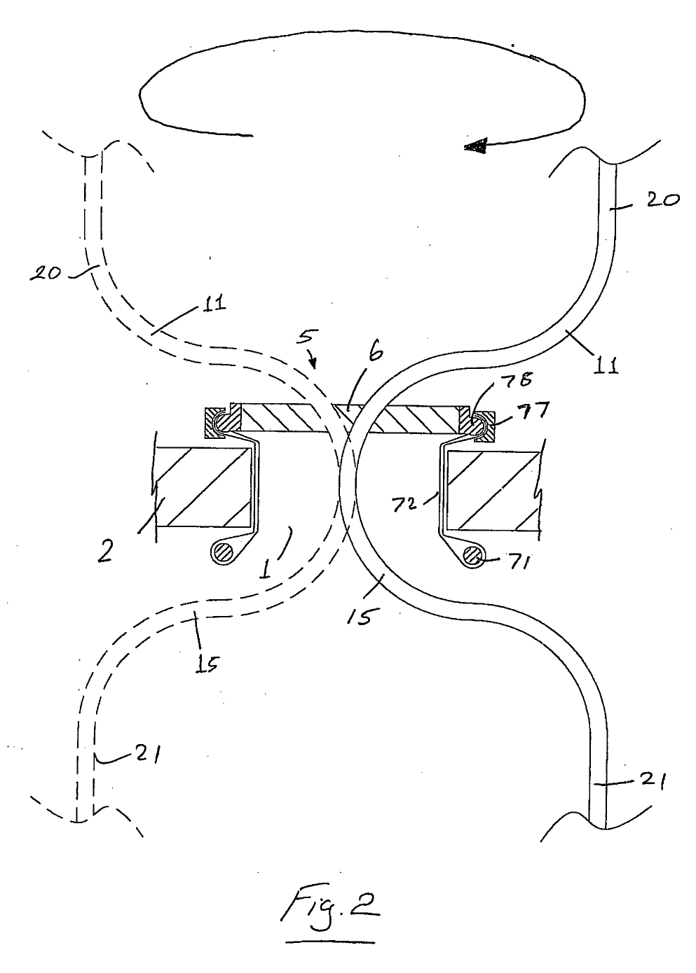

As will be apparent, especially from FIG. 2, by rotating the shaft 11 around the axis of the access valve 6, a surgeon can readily access a wide field through a relatively small access port 5.

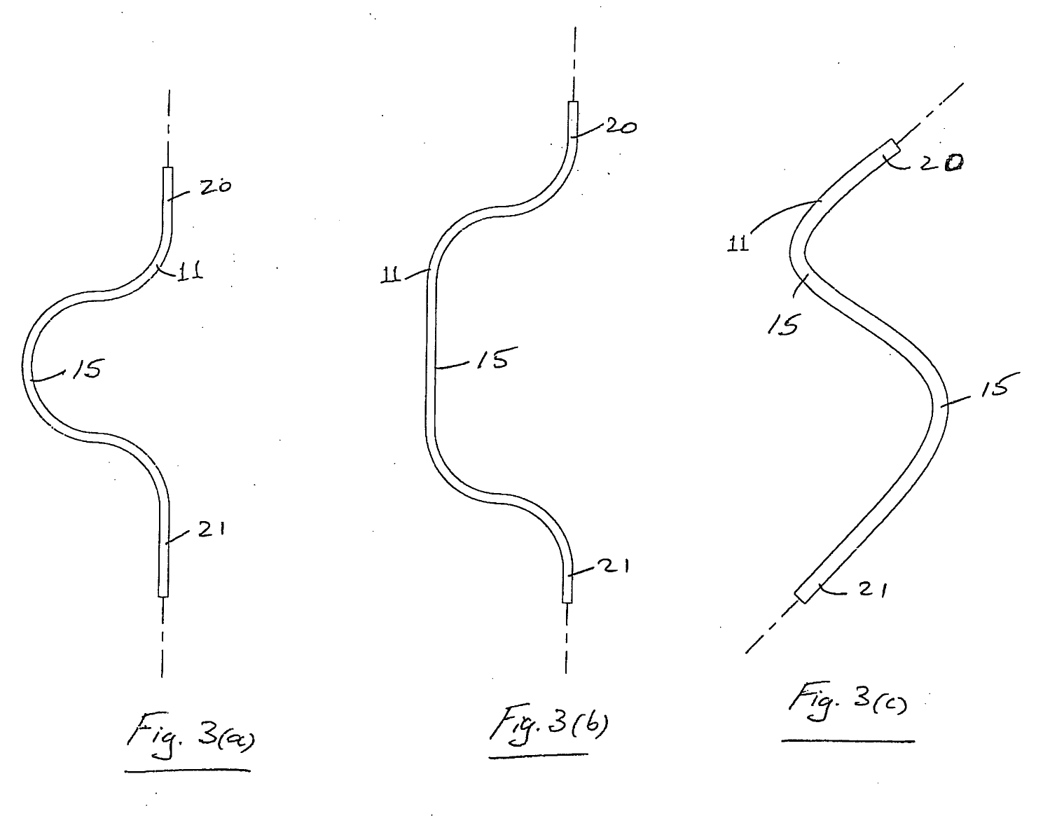

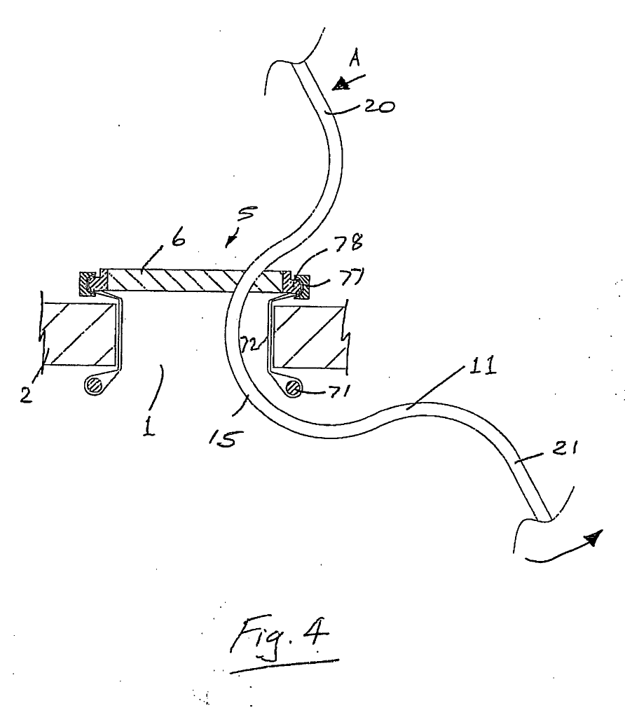

As will be apparent especially from FIGS. 3(a), 3(b) and 3(c) many different arrangements are possible. The bend section 15 may be of any desired shape such as arcuate or partially straight. The shaft shape may be pre-formed or the shaft 11 may be at least partially malleable or flexible for shaping in-situ. There may be one, two or any number of bends 15, some or all of which may be preformed.

Referring to FIG. 4 it will be noted that tilting of the shaft 11 gives even greater access. The shaft 11 may still be rotated, when tilted. In this way a surgeon has access to a relatively large area which can be many times wider than the area defined by the access valve/seal 6.

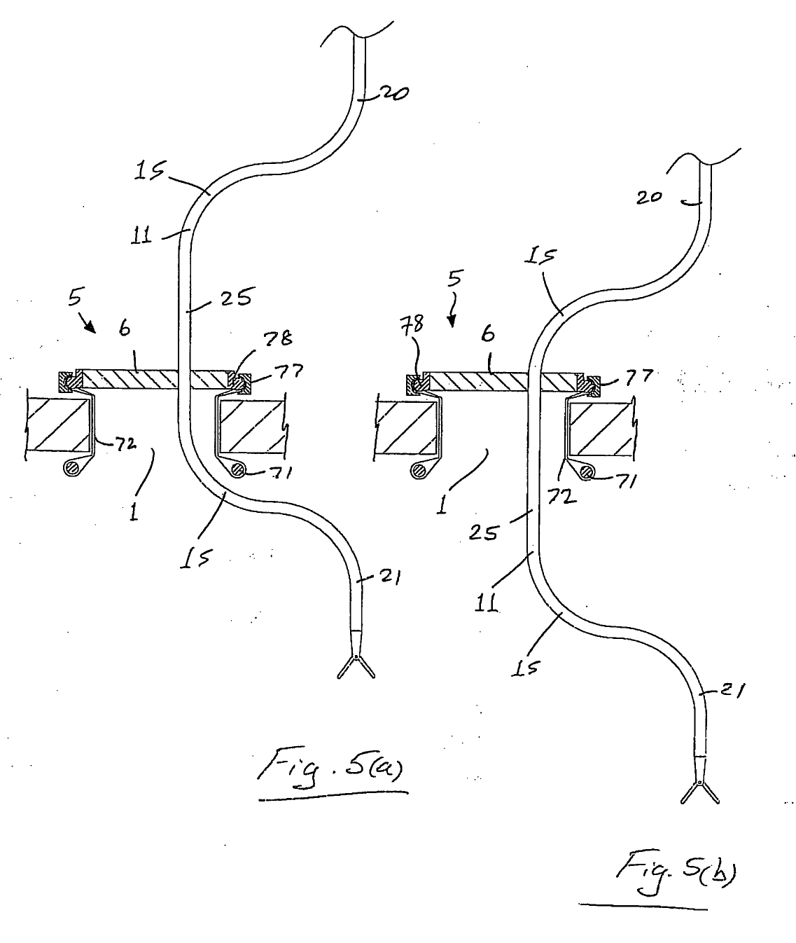

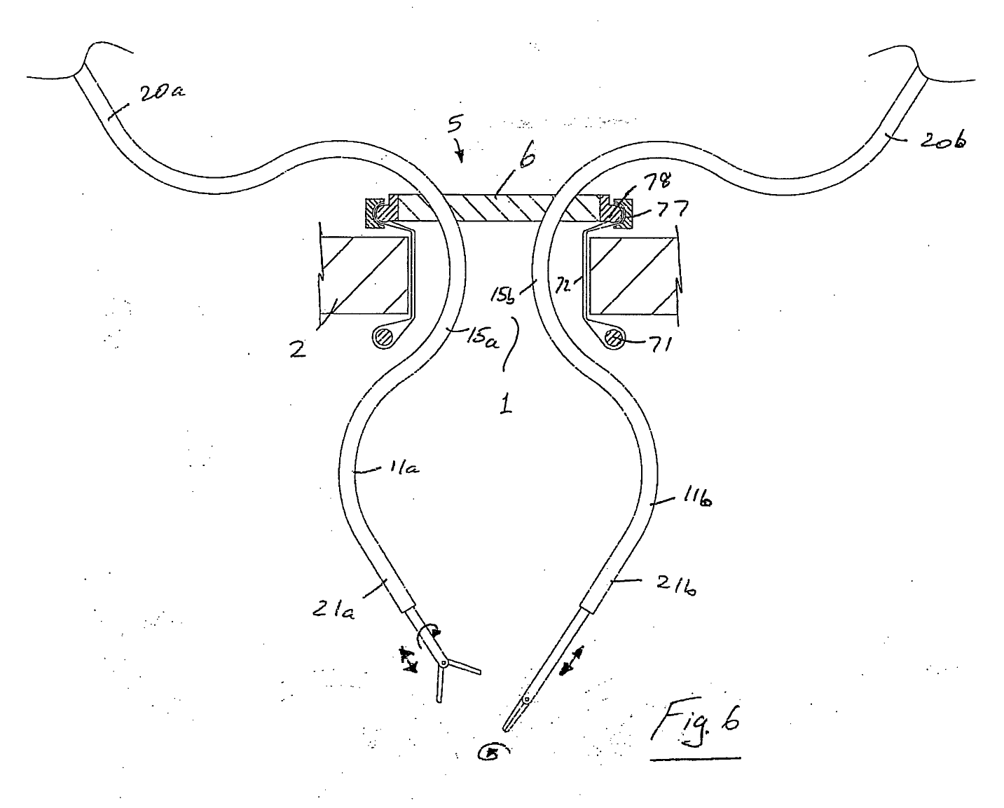

Referring to FIGS. 5(a) and 5(b), the shaft may have a straight section 25 to facilitate linear advancement through the access valve/seal 6.

FIG. 6 illustrates a system of the invention including two separate surgical devices identified as a and b. The system of the invention has a major advantage that a single port can be used to achieve triangulation. Using standard straight shafts this can only be achieved with two separate access ports. In the system of the invention instruments can be passed through bent cannulae. The bends 15a, 15b set up triangulation. The instruments may be flexible shaft instruments and can be readily advanced/retracted/rotated through the cannulae. The cannulae can also be manipulated/moved to access a range of different areas in the abdomen.

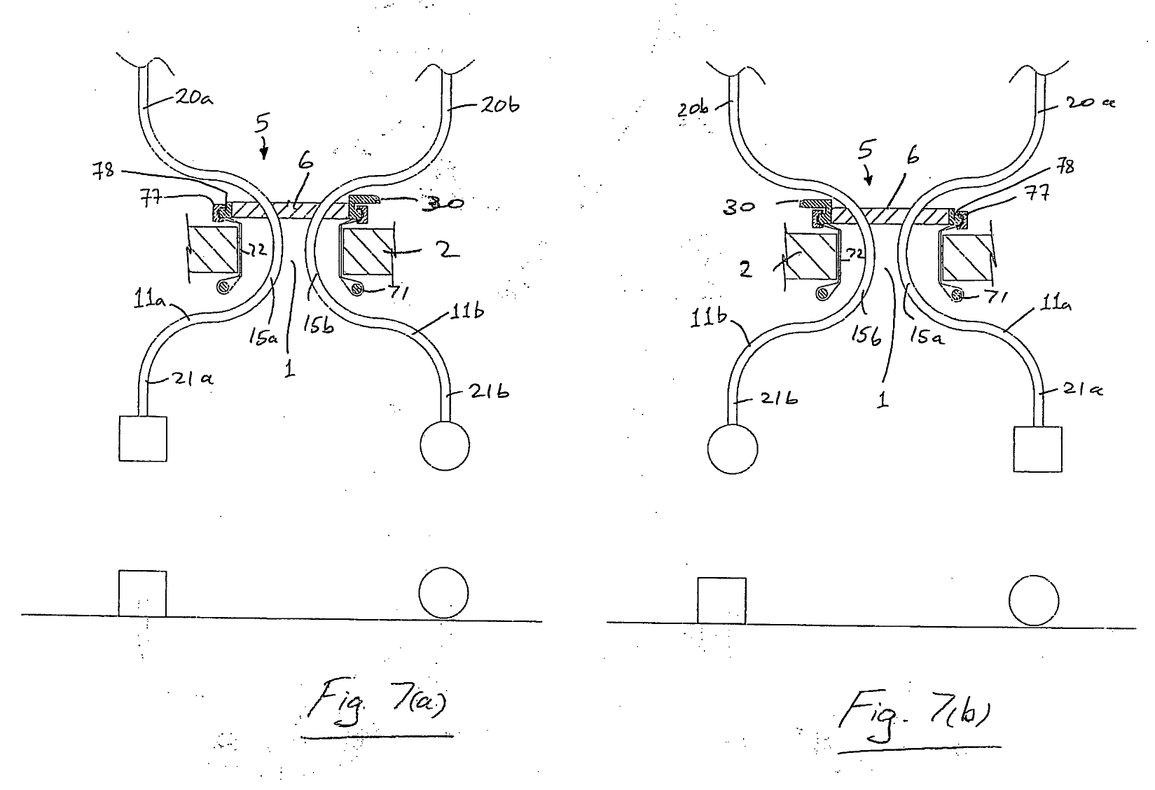

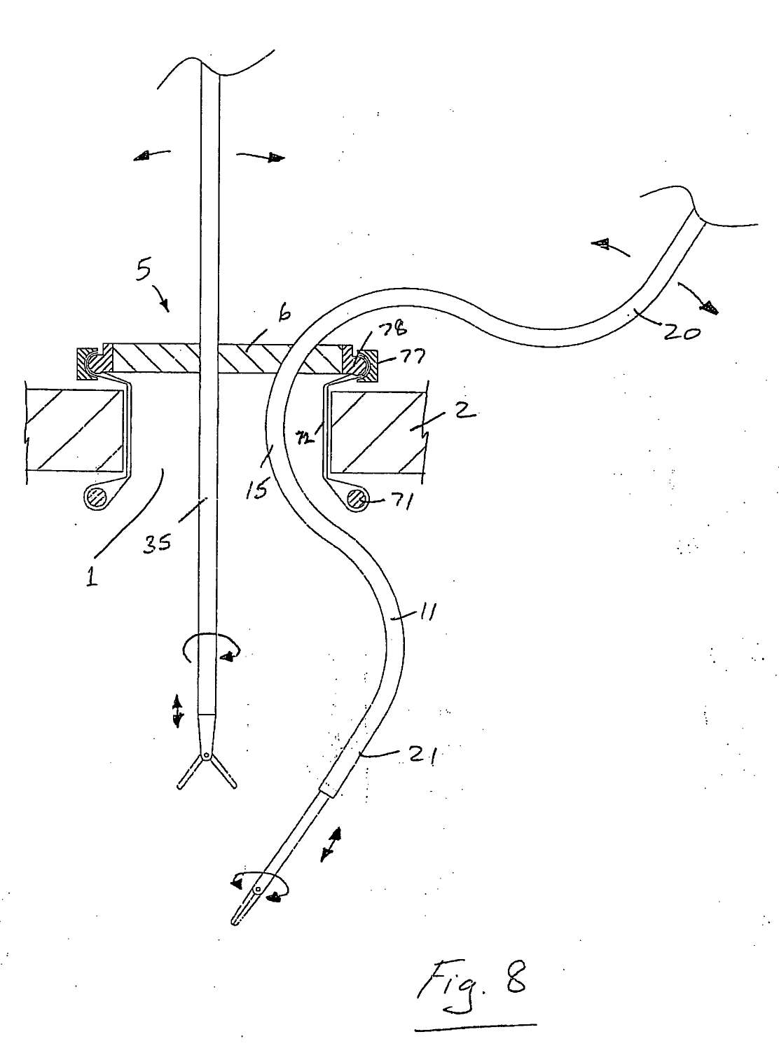

As illustrated in FIGS. 7(a) and 7(b), the system may facilitate rotation of the valve/seal 6 so that the instrument positions can be moved, and comparing FIGS. 7(a), 7(b) swopped by rotation through 180°. The valve/seal 6 has a handle 30 to facilitate the rotation.

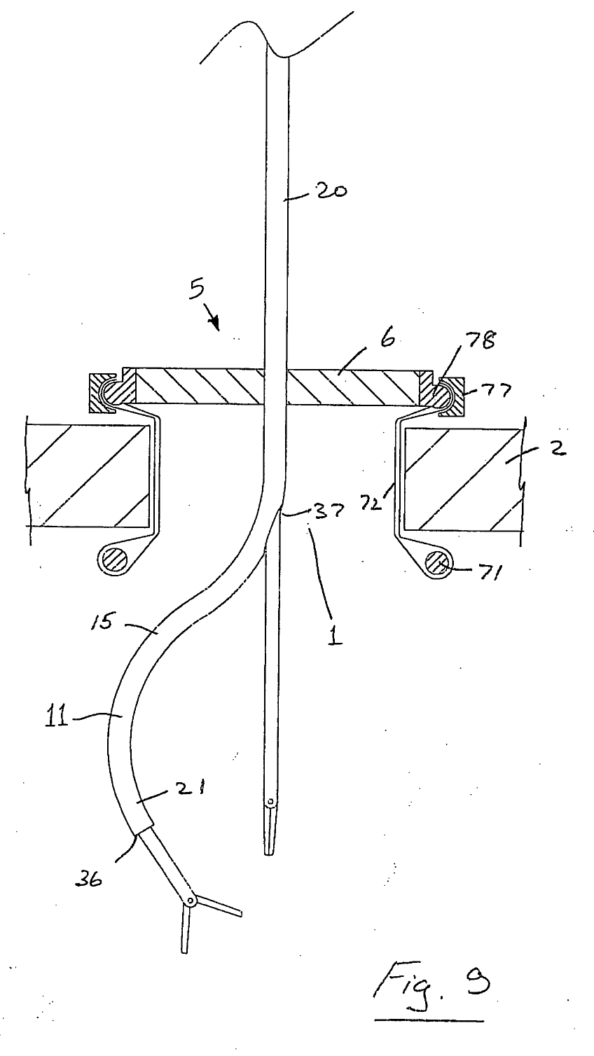

Referring to FIG. 8, the system may for example comprise a conventional straight device 35 and a bent instrument/cannula according to the invention. This arrangement allows triangulation to be achieved but with potentially less access. Another possible system (FIG. 9) involves the use of single curved cannula with two exit holes 36, 37 for instruments. The instruments may be similar or different. For example, the instrument advanced through the exit hole 37 may be a standard straight instrument, and the instrument advanced through the exit hole 36 may be a flexible instrument.

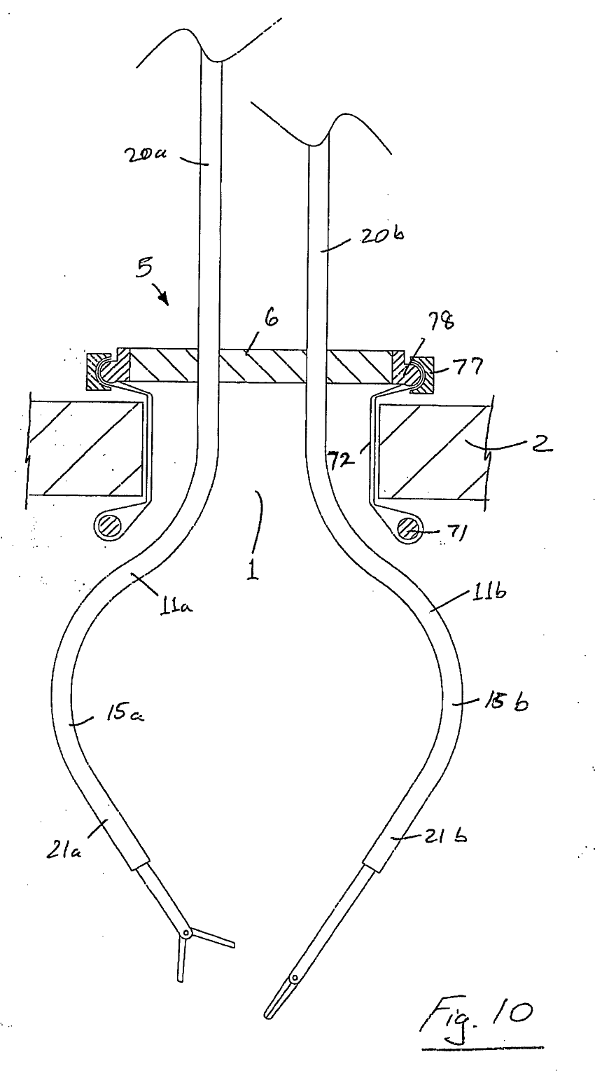

Many variations are possible. For example, in the arrangement illustrated in FIG. 10 the curved sections 15a, 15b may be located internally of the valve/seal 6 whilst still facilitating triangulation. Independent rotation, bending and/or vertical motion of each shaft is facilitated to provide enhanced access.

In another system of the invention illustrated in FIG. 11 one of the devices is a scope 40. This may be used in association with one or more instruments. In the case illustrated there are two instruments and a scope which are led through a single access port 5. This minimises the number of ports required and hence the number of incisions to be made with consequent improvements in patient trauma, suturing and healing time.

The end effector at the distal end of each instrument may be of the same type of end effector or different types of end effectors.

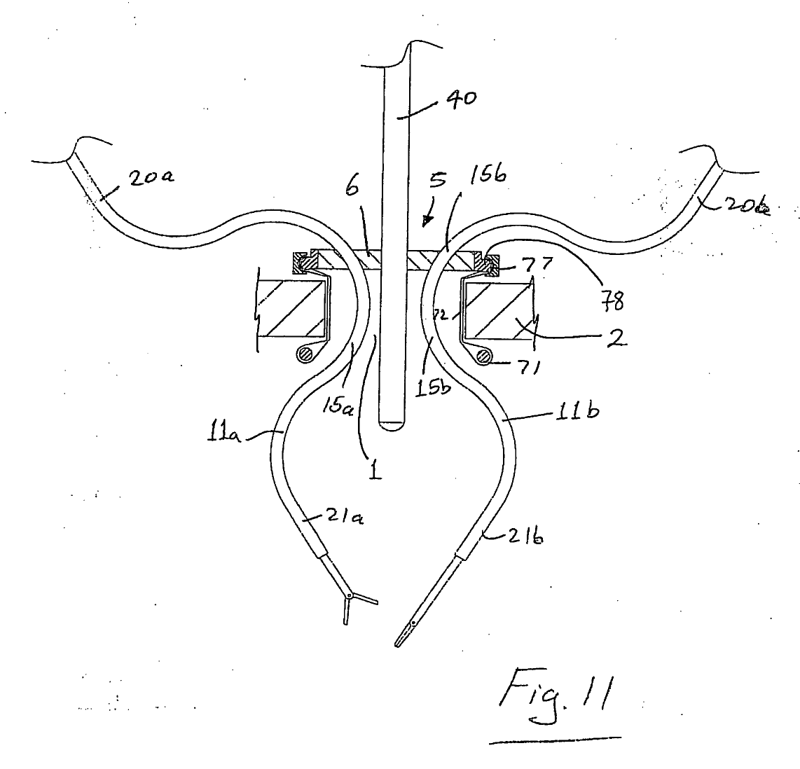

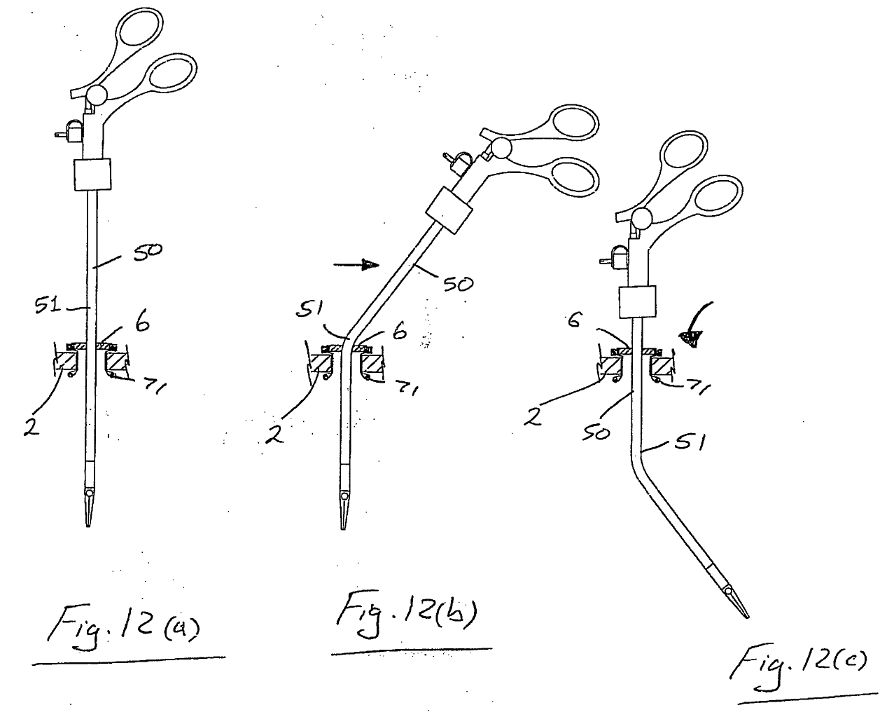

Referring to FIGS. 12(a) to 12(c), there is illustrated another system of the invention in which a device 50 with a bendable shaft 51 is inserted through the access valve/seal 6 (FIG. 12(a)).

Referring to FIG. 12(b) the surgeon bends the shaft 51 in-situ by pushing the handle off-axis. The shaft 51 bends against the valve housing. Thus, the device does not have to be withdrawn to provide a bend. The bending can be achieved using a single hand.

As illustrated in FIG. 12(c) the surgeon can now push the bent shaft 51 through the valve/seal 6 to gain access more easily in areas that standard straight laparoscopic devices cannot reach.

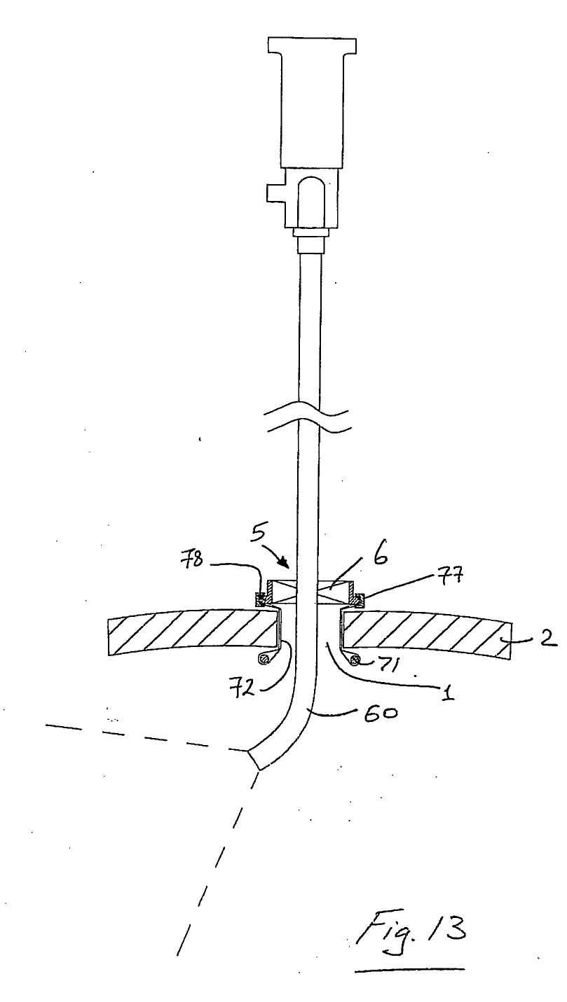

In a still further system illustrated in FIG. 13, a flexible laparoscope 60 is provided which is allowed to flex freely just beneath the peritoneum.

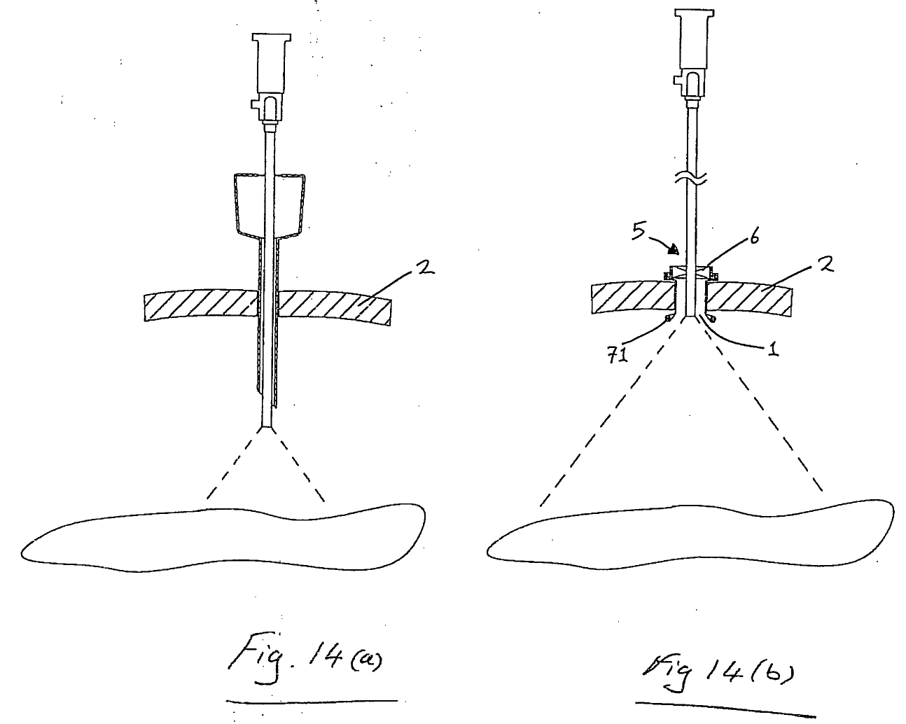

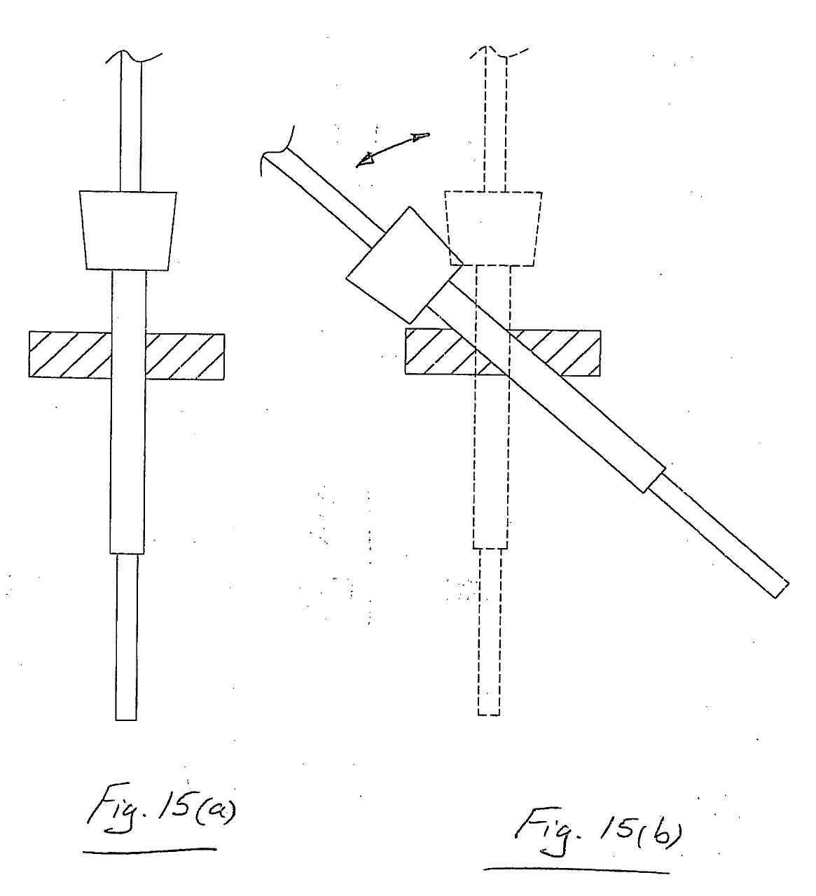

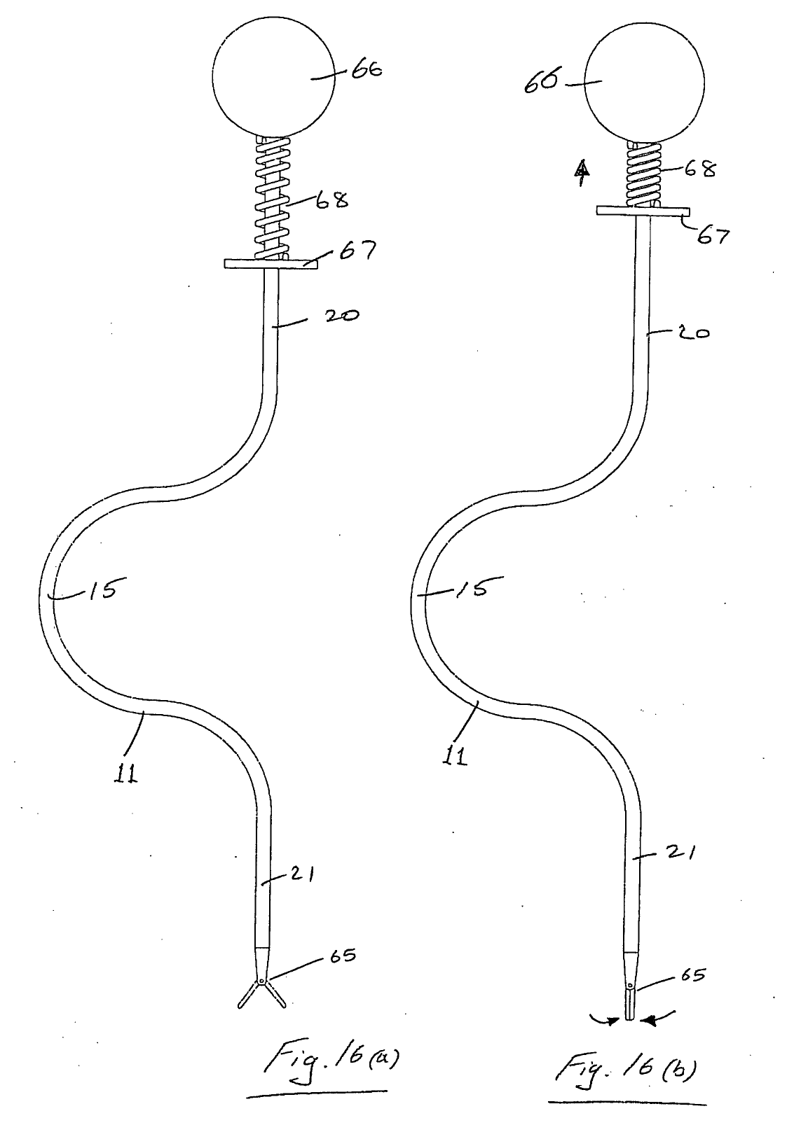

FIGS. 14(a) and 14(b) highlight the differences between the restricted field of vision which is achieved with a conventional trocar port (FIG. 14(a)), and the much wider field of vision that can be achieved using the system of the invention (FIG. 14(b)). The further disadvantages of a conventional trocar system will also be apparent from FIGS. 15(a) and 15(b). If a conventional trocar is tilted to one side it is biased to return to the static vertical position due to the resistance of the tissue surrounding the incision.

In contrast, with the surgical instrument 11 according to the invention, as described previously with reference to FIGS. 1 and 4, it is possible to manipulate the instrument 11 into a desired position within the wound interior by applying a manipulating force A to the proximal section 20 of the instrument 11 externally of the wound opening 1 (FIG. 4). Because of the bend section 15 in the instrument 11, the instrument 11 maintains the desired position within the wound interior even after the manipulating force A has been released. The instrument 11 will not be biased back to the original position.

Similarly it is possible to manipulate the instrument 11 into a desired orientation within the wound interior by applying a manipulating torque to the proximal section 20 of the instrument 11 externally of the wound opening 1, and the instrument 11 will maintain the desired orientation within the wound interior even after the manipulating torque has been released.

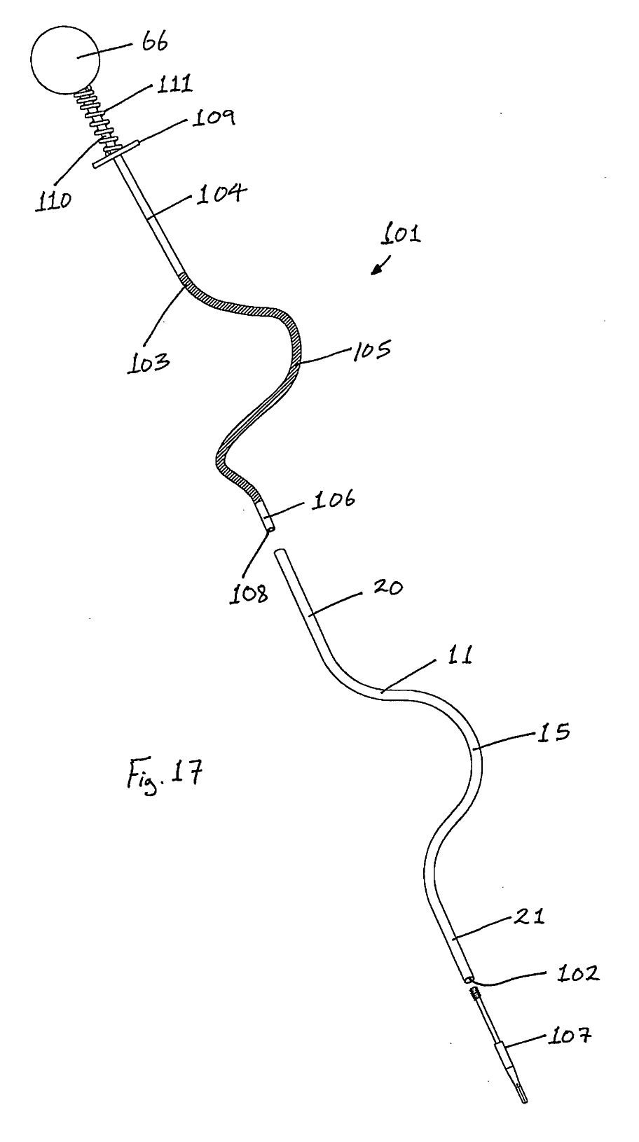

The systems of the invention may be used with either conventional or modified instrument manipulation. For example, referring to FIGS. 16(a) and 16(b), an end effector 65 of an instrument may be operated by an actuator comprising a ball-like handle 66 and a finger plate/bar 67 with a spring 68 therebetween. Using one hand the sphere handle 66 is located in the ball of a surgeon's hand and using his fingers the finger plate 67 is drawn upwardly against the action of the spring 68 to activate the end effector 65. Such an actuating system is generally easier and more comfortable to use than a standard pistol grip type actuator.

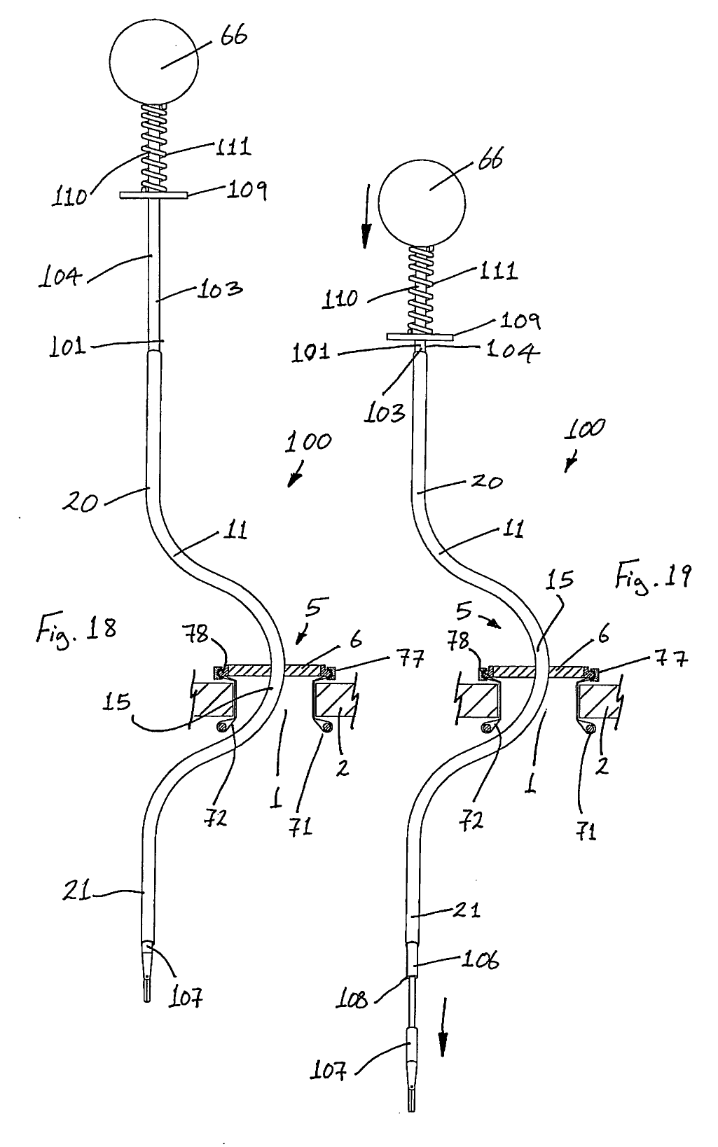

Referring to FIGS. 17 to 21 there is illustrated another surgical access system 100 according to the invention, which is similar to the systems described previously, and similar elements in FIGS. 17 to 21 are assigned the same reference numerals.

In this case the system 100 comprises the access port 5, as described above with reference to FIG. 1, the surgical device having the shaft 11 as described above with reference to FIG. 1, and a laparoscopic surgical instrument 101.

The surgical device comprises a rigid cannula, in this case, having a lumen 102 extending through the shaft 11.

The instrument 101 is similar to the instrument described above with reference to FIGS. 16(a) and 16(b). The instrument 101 has a shaft 103 with a rigid proximal region 104, a flexible intermediate region 105 and a rigid distal region 106. The instrument shaft 103 may be inserted through the cannula shaft 11, as illustrated in FIGS. 18 to 21.

The instrument 101 has a rigid end effector 107 which may be releasably coupled to the distal end 108 of the instrument shaft 103, for example by means of a screw-thread arrangement. An internal cable running through the instrument shaft 103 may also be coupled to the end effector 107.

An actuator 109 for actuating the end effector 107 is provided in the form of a plunger at the proximal end 110 of the instrument shaft 103. The actuator 109 is movable along the instrument shaft 103 parallel to the longitudinal axis of the instrument shaft 103 between an end effector open configuration (FIG. 16(a)) and an end effector closed configuration (FIG. 16(b)). A coiled spring 111 engages the actuator 109 to bias the actuator 109 towards the end effector open configuration.

FIGS. 18 and 19 illustrate axial advancement of the end effector 107 through the access port 5. It is noted that the bent cannula shaft 11 located in the access port 5 remains stationary upon advancement of the instrument 101.

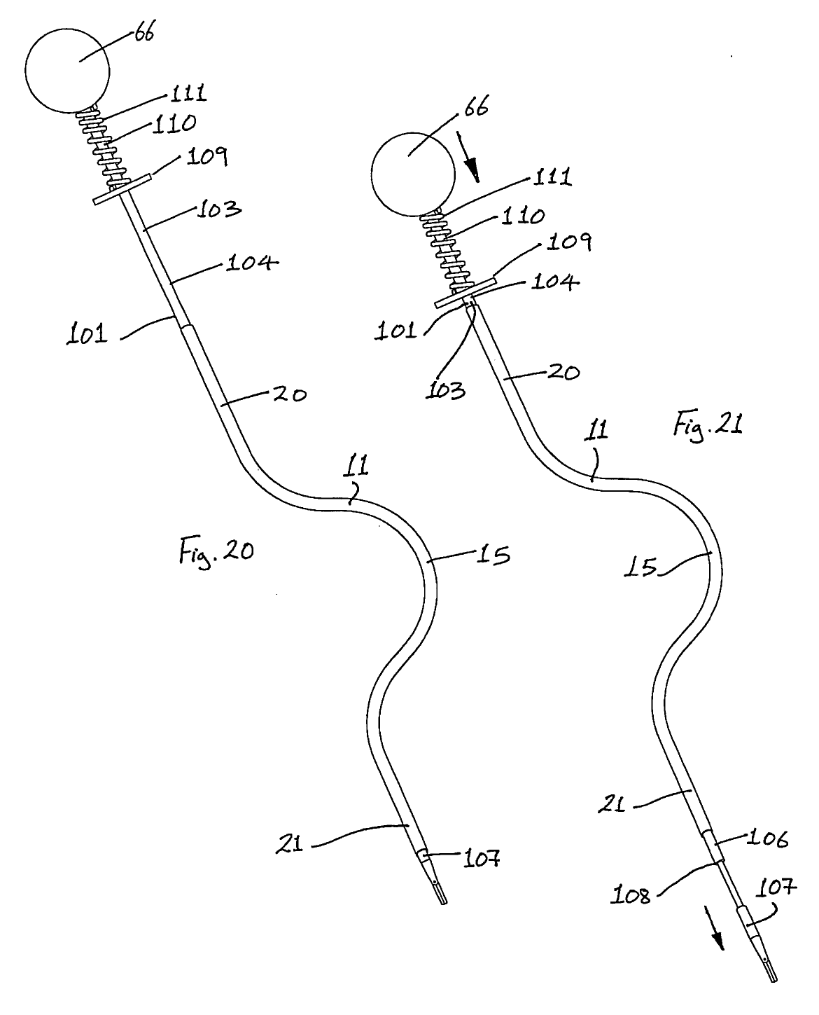

FIGS. 20 and 21 illustrate advancement of the end effector 107 into the abdomen towards a specific target.

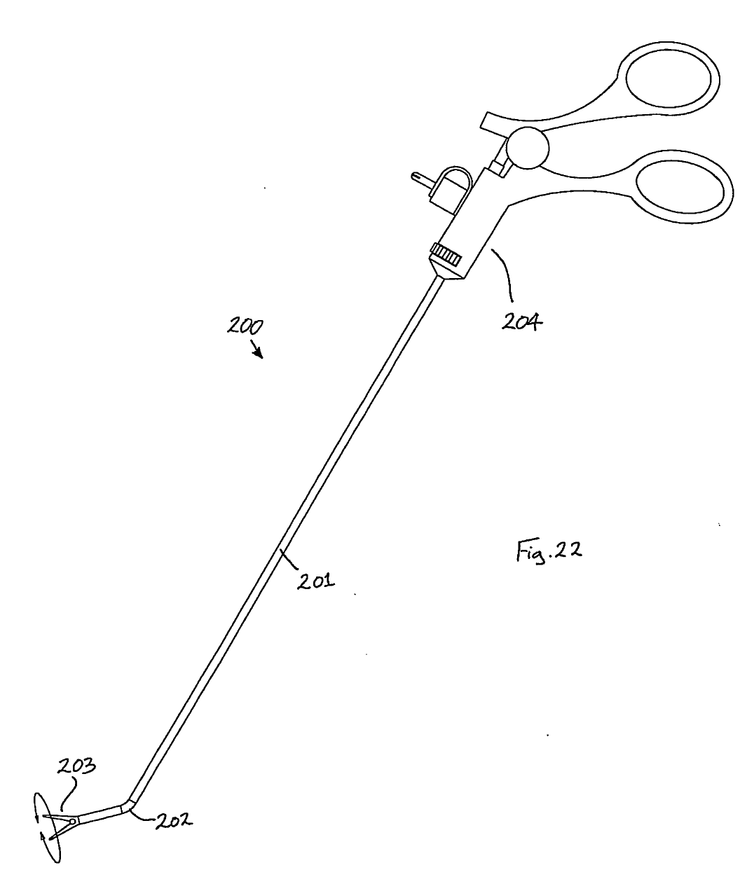

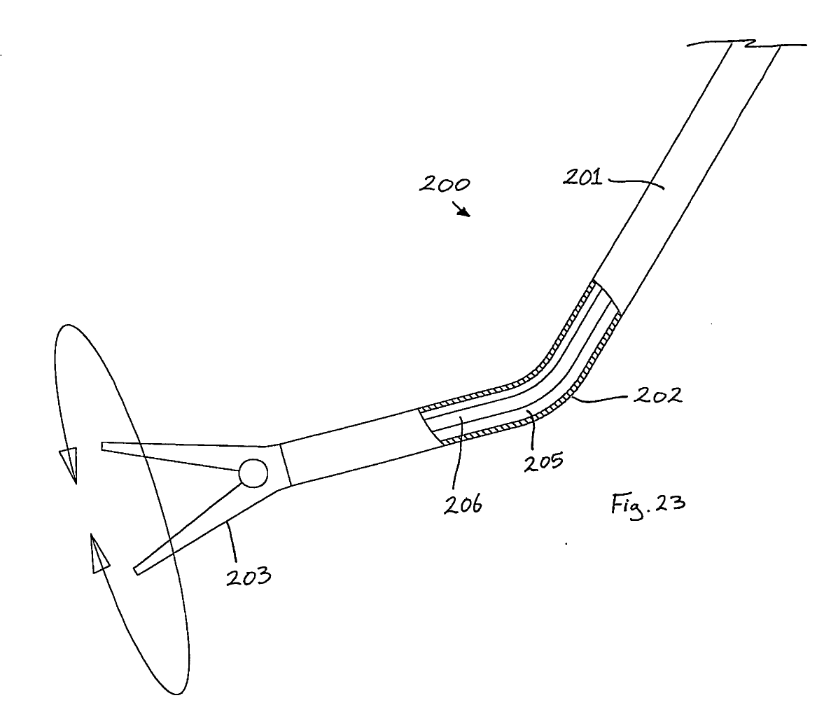

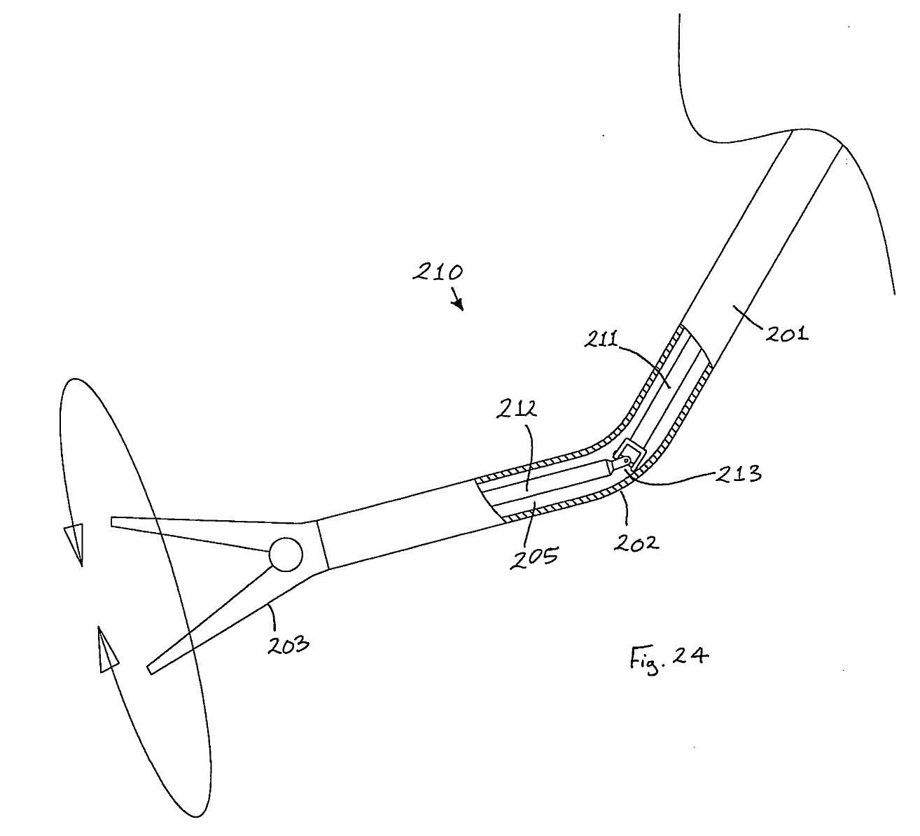

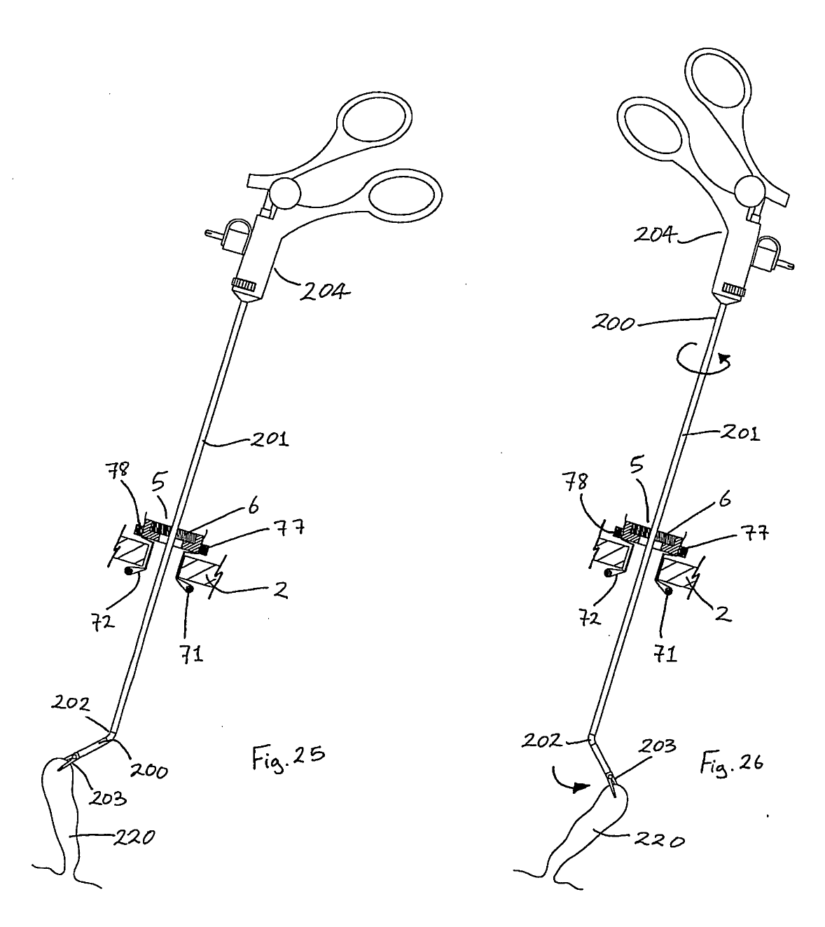

FIGS. 22 and 23 illustrate another surgical device 200 according to the invention, which is similar to the surgical device described previously with reference to FIG. 1, and similar elements in FIGS. 22 and 23 are assigned the same reference numerals.

In this case the surgical device 200 comprises a surgical instrument. The instrument 200 comprises a shaft 201 having a bend 202 therein, an end effector 203 at a distal end of the shaft 201, and an actuator 204 at a proximal end of the shaft 201 for actuating the end effector 203. As illustrated in FIG. 23, the shaft 201 defines a lumen 205 therethrough, and a tubular element 206 is located within the lumen 205 extending from the actuator 204 to the end effector 203 to couple the actuator 204 to the end effector 203.

The actuator 204 may be operated by a user to move the end effector 203 between an open configuration and a closed configuration, and/or to rotate the end effector 203 relative to the shaft 201.

FIG. 22 illustrates the bent laparoscopic instrument 200 with the finger wheel 204, the fixed bend 202, and the end effector 203 which spins when the finger wheel 204 is rotated. FIG. 23 illustrates one option to spin/actuate the end effector 203. The solid rod 206 actuates the bend effector 203 and transmits torque to spin the end effector 203.

It will be appreciated that in another embodiment of the invention, the end effector 203 may be translatable relative to the shaft 201, or may be translatable and rotatable relative to the shaft 201.

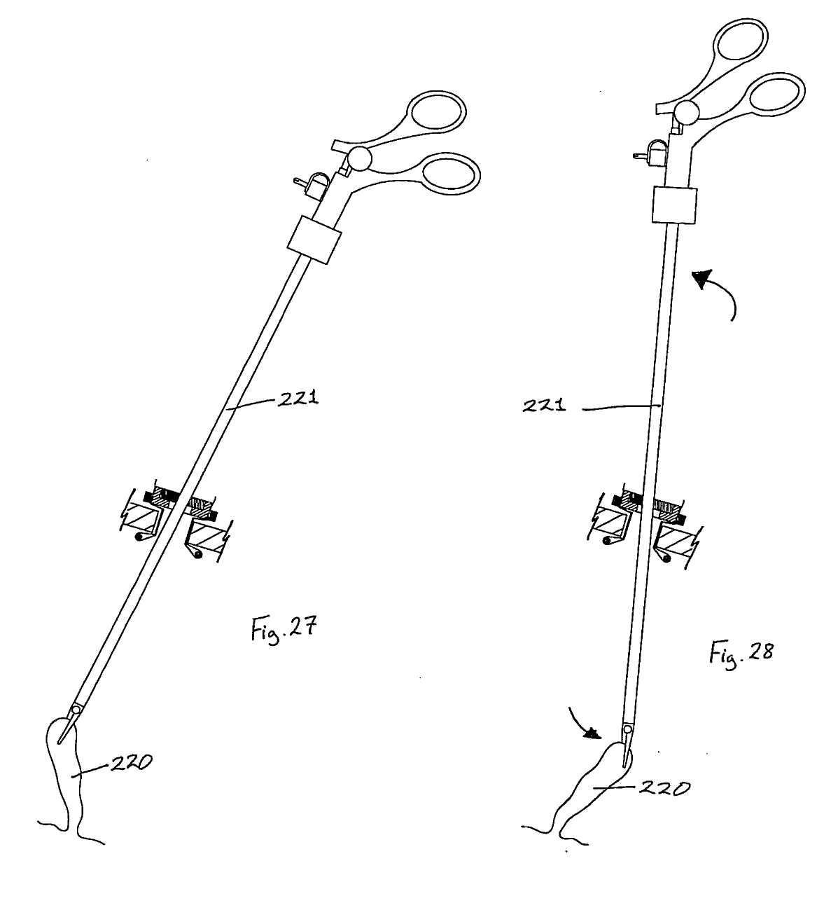

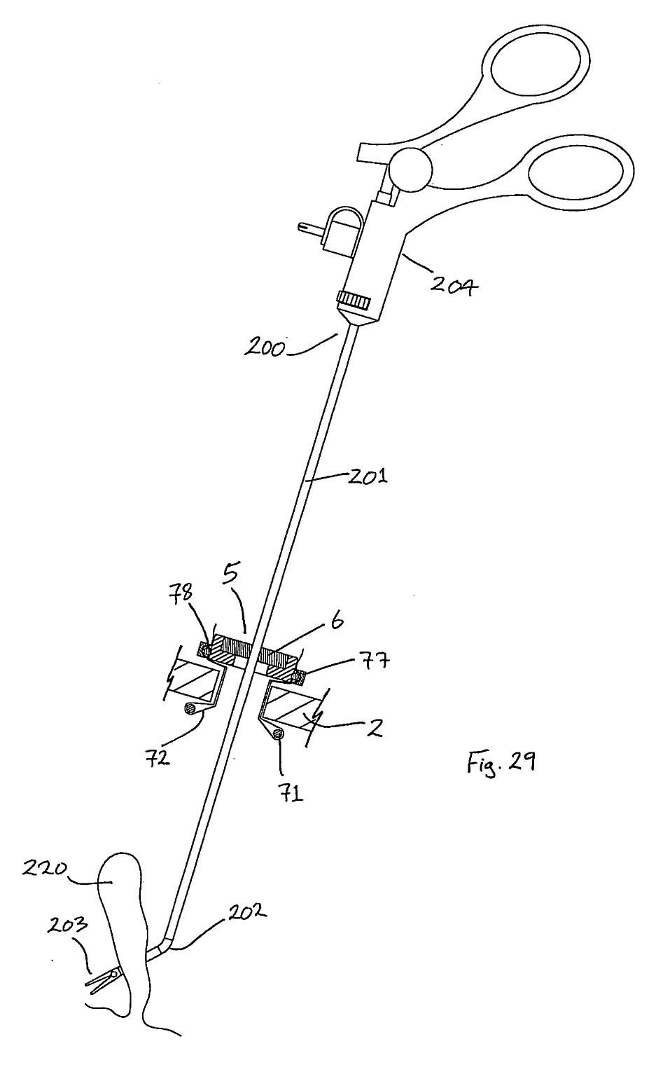

In FIG. 24 there is illustrated another surgical device 210 according to the invention, which is similar to the surgical device 200 of FIGS. 22 and 23, and similar elements in FIG. 24 are assigned the same reference numerals.

In this case the instrument 210 comprises a first tubular element 211 extending from the actuator 204 through the shaft lumen 205, and a second tubular element 212 extending from the end effector 203 through the shaft lumen 205. The first tubular element 211 is coupled to the second tubular element 212 by means of a universal joint 213.

FIG. 24 illustrates another option to actuate/spin the end effector 203 using the universal joint connection 213.

The bend 202 in the shaft 201 of the instrument 200 results in a number of advantages. For example, by simply rotating the proximal actuator 204, a piece of tissue 220 grasped by the end effector 203 may be swung to one side with a minimum of movement required by the surgeon exterior of the wound, as illustrated in FIGS. 25 and 26. This compares favourably with the excessive degree of movement required by the surgeon if it were attempted to swing the piece of tissue 220 to one side using a conventional instrument 221, as illustrated in FIGS. 27 and 28. In addition, the bend 202 in the shaft 201 of the instrument 200 may provide a simpler, easier means of accessing locations within the abdominal space, for example to access the space behind the piece of tissue 220, as illustrated in FIG. 29.

With the bend instrument 200, spinning the handle 204 main shaft 201 about its main axis causes the “off-set” end effetor 203 to sweep around. In this case the gall bladder 220 is easily moved around simply by spinning the instrument 200 (FIG. 25 and 26). No tilting is required. With a standard, straight laparoscopic instrument 221, to manipulate an organ 220 (e.g. a gall bladder), significant tilting of the instrument 221 is necessary (FIGS. 27 and 28). The bend instrument 200 allows the surgeon to easily pass around organs/vessels 220 etc. without undue tilting (e.g. FIG. 29).

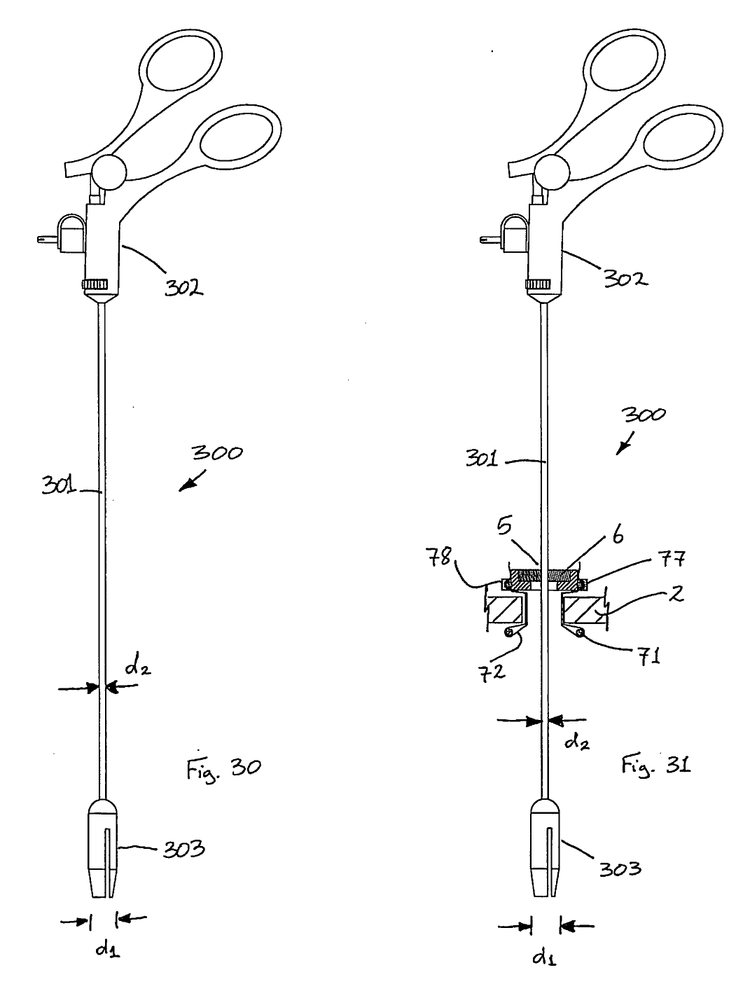

Referring to FIGS. 30 and 31 there is illustrated another surgical device 300 according to the invention. The surgical device 300 comprises a laparoscopic instrument.

The instrument 300 comprises a straight shaft 301, an actuator 302 at a proximal end of the shaft 301, and an end effector 303 at a distal end of the shaft 301. As illustrated in FIG. 30, the radial dimension d1 of the end effector 303 is substantially greater then the radial dimension d2 of the shaft 301.

As the end effector 303 is inserted through the gelatinous seal 6 in the access port 5, the seal 6 stretches to accommodate the larger radial dimension d1 of the end effector 303. When the end effector 303 has been fully inserted through the seal 6 into the abdominal cavity, the seal 6 seals around the smaller radial dimension d2 of the shaft 301 to prevent gas leakage from the insufflated abdominal cavity.

In one case the small diameter shaft 301 may have a diameter of 5 mm, and the large diameter end effector 303 may comprises a 12 mm diameter stapler.

The pin hole in the gel valve 6 can easily stretch to allow the end effector 303 to pass through. The small diameter shaft 301, during use of the instrument 300, results in less stress on the gel seal 6.

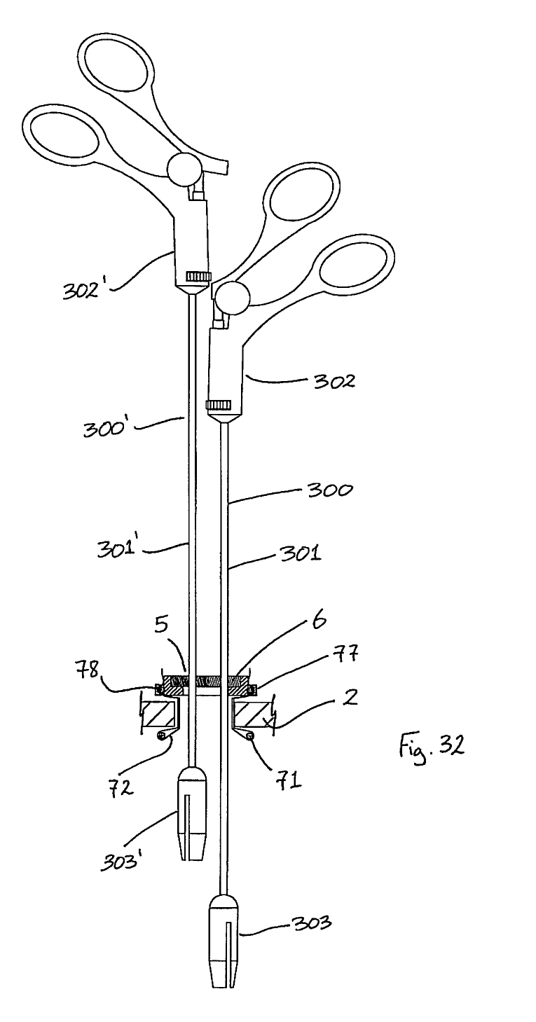

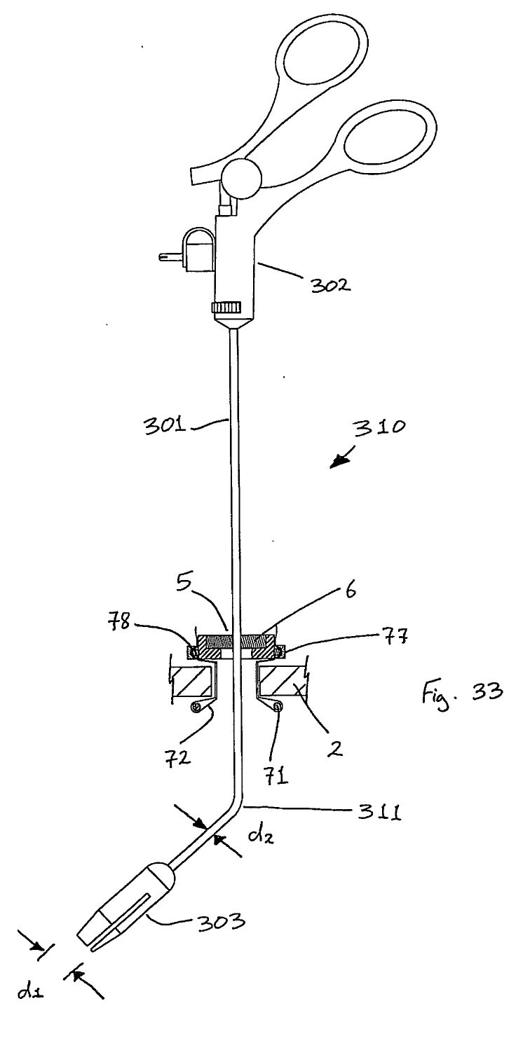

Because of the small diameter d2 of the shaft 301 of the instrument 300, two or more instruments 300, 3001 may be used through the same port 5 simultaneously (FIG. 32). This may not otherwise be possible if the diameter d2 of the shaft 301 were the same as the diameter d1 of the end effector 303.

In FIG. 33 there is illustrated a further surgical device 310 according to the invention, which is similar to the surgical device 300 of FIGS. 30 and 31, and similar elements in FIG. 33 are assigned the same reference numerals.

In this case the shaft 301 of the instrument 310 has a bend 311 therein.

With the surgical instrument of the invention the diameter of the shaft of the laparoscopic instrument is not necessarily dictated by the diameter of the end effector. The seal in the access port may accommodate a shaft diameter which is the same or is different to the end effector diameter.

The gel material of the seal is flexible enough to accommodate a range of diameters, for example 5 mm to 12 mm, passing through the seal while maintaining pneumoperitoneum.

The surgical access ports of the invention can be used in a number of ways. In one method the retractor is used as described above, the distal inner ring being inserted into an incision, and the outer ring being slid to controllably radially expand the incision. The retractor may then be locked in position. If necessary, the outer ring can be moved further downwardly to create a larger incision.

In some arrangements a device may be bent manually outside the body and the bent device is delivered through the access port to readily access the operative site.

In a further embodiment a device is inserted into the access port and the surgeon uses the abdominal wall itself to bend the shaft and then insert the bent section further into the abdomen.

The access ports of the invention have at least some of the following advantages:

Controlled Radial Expansion
1. Greater access using smaller incision
2. Can vary incision size as need be (e.g. specimen removal during lap coli.)

Greater Sealing Capabilities
1. No gas leakage from the wound margins
2. Cannot be inadvertently pulled out of the incision
3. Will seal any incision and never require secondary sealing method (suture, Hassan port, etc.)

Eliminate Intra-Abdominal Profile
1. Gives back more working space in the abdomen (critical in pelvic surgery)
2. Perineal access for operations such as Radical Prostatectomy.

Protection of Wound from Infection and Cancer Seeding
1. Tight seal with no “chimney stack” effect
2. Upon removal all areas of potential contamination are isolated from the incision

Reduced Extra-Abdominal Profile
1. Will increase the effective working length of an instrument
2. Greater working area outside the abdomen

Increase the Freedom of Movement of Conventional Laparoscopic Instruments

The systems of the invention can be used in a wide range of laparoscopic surgical procedures, for example, gall bladder removal. In this case a single access port is inserted as described above. Two instruments may be inserted through the valve seal. One instrument is used to hold the liver whilst a second bent instrument is used to cut one side of the gall bladder, then moved as described above to cut the other side of the gall bladder.

The systems can also be used for carrying out a laparoscopic colonectomy, or a hernia repair, for example.

The invention is not limited to the embodiments hereinbefore described, with reference to the accompanying drawings, which may be varied in construction and detail.

1. A surgical device comprising a shaft, the shaft comprising a distal section, a proximal section, and a bend section located between the distal section and the proximal section.

2. A device as claimed in claim 1 wherein the distal section is substantially parallel to the proximal section.

3. A device as claimed in claim 1 wherein the distal section and proximal section are substantially co-axial.

4. A device as claimed in claim 1 wherein the distal section and the proximal section are substantially offset.

5. A device as claimed in claim 1 wherein the distal section is substantially straight.

6. A device as claimed in claim 1 wherein the proximal section is substantially straight.

7. A device as claimed in claim 1 wherein the bend section is at least partially of curved shape.

8. (canceled)

9. A device as claimed in claim 1 wherein the bend section is pre-set.

10. A device as claimed in claim 1 wherein the shaft is at least partially rigid.

11. (canceled)

12. (canceled)

13. A device as claimed in claim 1 wherein the shaft is at least partially malleable.

14. A device as claimed in claim 1 wherein the shaft is at least partially flexible.

15. (canceled)

16. A device as claimed in claim 1 wherein the shaft defines a lumen extending therethrough.

17. (canceled)

18. A device as claimed in claim 1 wherein the surgical device comprises an instrument.

19. A device as claimed in claim 18 wherein the surgical device comprises an end effector at a distal end of the shaft.

20. A device as claimed in claim 19 wherein the end effector is releasably coupled to the distal end of the shaft.

21. A device as claimed in claim 19 wherein the radial dimension of the end effector is substantially greater than the radial dimension of the shaft.

22. A device as claimed in claim 19 wherein the end effector is movable between an open configuration and a closed configuration.

23. A device as claimed in claim 19 wherein the end effector is movable relative to the shaft.

24.-26. (canceled)

27. A device as claimed in claim 19 wherein the surgical device comprises an actuator for actuating the end effector.

28.-32. (canceled)

33. A device as claimed in claim 27 wherein the surgical device comprises a coupling member to couple the actuator to the end effector.

34.-39. (canceled)

40. A laparoscopic surgical device as claimed in claim 1.

41. A surgical instrument comprising a shaft, an end effector at a distal end of the shaft, and an actuator for actuating the end effector, the actuator being movable along the shaft between an end effector open configuration and an end effector closed configuration.

42. An instrument as claimed in claim 41 wherein the actuator is movable parallel to the longitudinal axis of the shaft.

43. An instrument as claimed in claim 41 wherein the actuator comprises a plunger.

44. An instrument as claimed in claim 41 wherein the actuator is biased towards an end effector open configuration.

45. An instrument as claimed in claim 41 wherein the end effector is releasably coupled to the distal end of the shaft.

46. An instrument as claimed in claim 41 wherein the end effector is movable between an open configuration and a closed configuration.

47. An instrument as claimed in claim 41 wherein the end effector is movable relative to the shaft.

48.-50. (canceled)

51. An instrument as claimed in claim 41 wherein the shaft is at least partially flexible.

52. An instrument as claimed in claim 41 wherein the shaft is at least partially malleable.

53. An instrument as claimed in clam 41 wherein the shaft is at least partially rigid.

54. An instrument as claimed in claim 41 wherein a distal region of the shaft adjacent a distal end of the shaft is rigid.

55. An instrument as claimed in claim 41 wherein a proximal region of the shaft adjacent a proximal end of the shaft is rigid.

56. An instrument as claimed in claim 41 wherein an intermediate region of the shaft intermediate the proximal region and the distal region is flexible.

57. An instrument as claimed in claim 41 wherein the shaft of the instrument is configured for insertion through a surgical device shaft.

58. A surgical instrument comprising a shaft and an end effector at a distal end of the shaft, the radial dimension of the end effector being substantially greater than the radial dimension of the shaft.

59. A laparoscopic surgical instrument as claimed in claim 41.

60. A surgical access system comprising:
a surgical access port configured for location adjacent to an incision; and
a surgical device as claimed in claim 1 for insertion through the access port.

61. A system as claimed in claim 60 wherein the access port comprises an access valve or seal, through which the surgical device is insertable.

62. A system as claimed in claim 60 wherein the access valve or seal comprises a gelatinous elastomeric material for receiving the surgical device.

63. (canceled)

64. A system as claimed in claim 60 wherein the access port comprises a retractor.

65. (canceled)

66. A system as claimed in claim 64 wherein the retractor comprises:
a distal member for insertion through an incision; and
an elongate member extending proximally from the distal member.

67.-76. (canceled)

77. A system as claimed in claim 60 wherein the surgical device comprises a first surgical instrument.

78. (canceled)

79. A system as claimed in claim 60 wherein the system comprises a second surgical device for insertion through the access port.

80.-84. (canceled)

85. A system as claimed in claim 60 comprising a third surgical device for insertion through the access port.

86. (canceled)

87. A system as claimed in claim 60 wherein the system comprises a surgical instrument comprising a shaft, the instrument shaft being insertable through the surgical device shaft.

88. (canceled)

89. A laparoscopic surgical access system as claimed in claim 60.

90. A method of performing a surgical procedure, the method comprising the steps of:
creating a wound opening;
inserting a surgical device at least partially through the wound opening to access the wound interior;
applying a manipulating action to the surgical device to manipulate the surgical device into a desired position and/or orientation within the wound interior; and
releasing the manipulating action, the surgical device substantially maintaining the desired position and/or orientation within the wound interior after release of the manipulating action.

91. A method as claimed in claim 90 wherein the manipulating action comprises a manipulating force to manipulate the surgical device into a desired position.

92. A method as claimed in claim 90 wherein the manipulating action comprises a manipulating torque to manipulate the surgical device into a desired orientation.

93. A method as claimed in claim 90 wherein the method comprises the step of sealing the wound opening.

94. A method as claimed in claim 90 wherein the method comprises the step of retracting the wound opening.

95. A method as claimed in claim 90 wherein the surgical device comprises a surgical instrument.

96. A method as claimed in claim 90 wherein the surgical device comprises a shaft, the shaft comprising a bend section.
